(19) United States
(12) Patent Application Publication
(10) Pub. No.: US 2018/0344140 A1
(43) Pub. Date: Dec. 6, 2018

(54) ILLUMINATOR WINDOW FOR A MULTIPLE VIEWING ELEMENT ENDOSCOPE

(71) Applicant: EndoChoice, Inc., Alpharetta, GA (US)

(72) Inventor: Amram Aizenfeld, Ramot Menashe (IL)

(73) Assignee: EndoChoice, Inc., Alpharetta, GA (US)

(21) Appl. No.: 15/991,939

(22) Filed: May 29, 2018

Related U.S. Application Data
(60) Provisional application No. 62/512,472, filed on May 30, 2017.

(51) Int. Cl. A61B 1/06; A61B 1/00; A61B 1/05

(52) U.S. Cl. CPC A61B 1/0676; A61B 1/00096; A61B 1/0011; A61B 1/12; A61B 1/00117; A61B 1/05; A61B 1/00009; A61B 1/00163

(57) ABSTRACT

The present specification describes an endoscope tip having an illuminator window for covering an illuminator cavity holding an illuminator attached with a circuit board installed in the endoscope tip. The illuminator window has a rectangular shape with a distal long surface facing away from the illuminator, a proximal long surface facing towards the illuminator, and a first and a second side surface. The cavity has a first side wall positioned at a predefined distance from the first side surface of the window creating a first gap and a second side wall positioned at a predefined distance from the second side surface of the window creating a second gap. The first side surface of the window is attached to the first sidewall by application of glue in the first gap and the second side surface of the window is attached to the second sidewall by application of glue in the second gap, wherein the width of the first and the second gaps is greater than 0.8 mm and the distance between the illuminator an the window is less than or equal to 0.4 mm.

### CROSS-REFERENCE TO RELATED APPLICATIONS

This application claims the benefit of priority under 35 U.S.C. 119 of U.S. Provisional Patent Application No. 62/512,472, filed on May 30, 2017, which is incorporated by reference herein in its entirety.

### FIELD

The present specification relates generally to endoscopes, and more specifically, to an illuminator window fitted onto an illuminator cavity within the tip section of a multiple viewing elements endoscope.

### BACKGROUND

An endoscope is a medical instrument used for examining and treating internal body cavities such as the alimentary canals, airways, the gastrointestinal system, and other organ systems. Conventional endoscopes are usually an elongated tubular shaft, rigid or flexible, having a video camera and a fiber optic light guide for directing light from an external light source situated at a proximal end of the tube to a distal tip. Also, most endoscopes are provided with one or more channels, through which medical devices, such as forceps, probes, and other tools, may be passed. Further, during an endoscopic procedure, fluids, such as water, saline, drugs, contrast material, dyes, or emulsifiers are often introduced or evacuated via the shaft. A plurality of channels, one each for introduction and suctioning of liquids, may be provided within the shaft.

Endoscopes have attained great acceptance within the medical community, since they provide a means for performing procedures with minimal patient trauma, while enabling the physician to view the internal anatomy of the patient. Over the years, numerous endoscopes have been developed and categorized according to specific applications, such as cystoscopy, colonoscopy, laparoscopy, upper gastrointestinal (GI) endoscopy among others. Endoscopes may be inserted into the body's natural orifices or through an incision in the skin.

Some endoscopes have viewing elements for viewing an internal organ, such as the colon, and an illuminator for illuminating the field of view of the viewing elements. The viewing elements and illuminators are located in a tip of the endoscope and are used to capture images of the internal walls of the body cavity being endoscopically scanned. The captured images are sent to a control unit coupled with the endoscope via one of the channels present in the scope shaft, for being displayed on a screen coupled with the control unit. During an endoscopic procedure an operating physician guides the endoscope within a patient's body by using the captured images displayed on the screen coupled with the control unit as a guide.

Usually the illuminators used for illuminating the field of view of the viewing elements are coupled with illuminator circuit boards and placed in illuminator cavities adjacent to the viewing elements. Each illuminator cavity is covered with a glass window which is attached to the side walls of the cavity by means of glue. In conventional window and cavity designs the gap between a side of the window and a corresponding side wall of the cavity is very small of the order of 0.05 mm which results in a minimal quantity of glue to be applied. Hence the window is susceptible to become detached from the cavity during operation or cleaning of the endoscope. Further, due to conventional design of the window and the cavity, it is difficult to align the window in the center of the cavity. Also, due to the distance between the illuminator and the window in the known designs, portions of the light emitted by the illuminator are lost in transit and not transmitted outside the window.

Hence, there is need for an improved design of the illuminator window and illuminator cavity which enables a sturdy attachment of the window with the cavity walls and also does not cause reduction in brightness of the light transmitted out of the window.

### SUMMARY

In some embodiments, the present specification disclosed an endoscope tip comprising an illuminator window for covering an illuminator cavity comprising an illuminator coupled with a circuit board installed in the endoscope tip, the illuminator window comprising a distal long surface facing away from the illuminator, a proximal long surface facing towards the illuminator, and a first and a second side surface, the cavity comprising a first side wall positioned at a predefined distance from the first side surface of the window creating a first gap and a second side wall positioned at a predefined distance from the second side surface of the window creating a second gap, the first side surface of the window being attached to the first sidewall by application of glue in the first gap and the second side surface of the window being attached to the second sidewall by application of glue in the second gap, wherein the width of the first and the second gaps is greater than 0.8 mm and the distance between the illuminator and the window is less than or equal to 0.4 mm.

Optionally, the illuminator window is rectangular in shape, wherein the length of the distal surface is equal to the length of the proximal surface, and the length of the first side surface is equal to the length of the second side surface.

Optionally, the length of the distal and proximal surfaces is within a predetermined range.

Optionally, the width of the first and the second gap is 0.3 mm.

Optionally, the width of the first and the second gap is within a predetermined range.

Optionally, the cavity comprises a window support element for enabling the window to be placed in the center of the cavity.

Optionally, the window support element is a metal ring having a predefined diameter.

Optionally, the window support element enables placement of the illuminator window at a predefined distance from the illuminator.

Optionally, the distal long surface of the window is truncated to create a first slanting side and a second slanting side, thereby increasing the width of the first and the second gap respectively, the length of the truncated distal surface being shorter than the length of the proximal long surface.

Optionally, the increased width of the first and the second gap is within a predetermined range.

In some embodiments, the present specification discloses an illuminator window for covering an illuminator cavity comprising an illuminator coupled with a circuit board installed in an endoscope tip, the illuminator window comprising a distal surface facing away from the illuminator, a proximal surface facing towards the illuminator, length of the proximal surface being greater than length of the distal surface, and a first and a second slanting side surface, the cavity comprising a first side wall positioned at a predefined distance from the first side surface of the window creating a first gap and a second side wall positioned at a predefined distance from the second side surface of the window creating a second gap, the first side surface of the window being attached to the first sidewall by application of glue in the first gap and the second side surface of the window being attached to the second sidewall by application of glue in the second gap, wherein the width of the first and the second gaps is greater than 0.8 mm and the distance between the illuminator and the window is less than or equal to 0.4 mm.

Optionally, the distal surface is parallel to the proximal surface.

Optionally, the window is shaped as a parallelogram.

Optionally, a length of the first slanting side surface is equal to the length of the second slanting side surface, said length being smaller than lengths of the distal and proximal surfaces.

Optionally, a length of the distal surface and proximal surface are within predetermined ranges.

Optionally, a width of the first and the second gap is 0.3 mm.

Optionally, the width of the first and the second gap are within predetermined ranges.

Optionally, the cavity comprises a window support element for enabling the window to be placed in the center of the cavity.

Optionally, the window support element is a metal ring having a diameter within a predetermined range.

Optionally, the window support element enables placement of the illuminator window at a predefined distance from the illuminator.

The aforementioned and other embodiments of the present specification shall be described in greater depth in the drawings and detailed description provided below.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1 is an illustration of a multiple viewing element endoscopy system;

FIG. 2 is a cross-sectional view of a tip section of a multiple viewing element endoscope;

FIG. 3 is an isometric view of a typical multiple viewing element endoscope tip cover;

FIG. 4 is an illustration of a conventional illuminator coupled with an optical assembly within a tip section of an endoscope;

FIG. 5A is an illustration showing illuminator windows affixed to illuminator cavities in an endoscope tip, in accordance with an embodiment of the present specification;

FIG. 5B is an exploded cross-sectional view of the illuminator window affixed to the illuminator cavity shown in FIG. 5A;

FIG. 6 is a block diagram of an illuminator window affixed to an illuminator cavity in an endoscope tip, in accordance with an embodiment of the present specification; and

FIG. 7 is a cross-sectional view of an illuminator window affixed to an illuminator cavity, in accordance with an embodiment of the present specification.

### DETAILED DESCRIPTION

In embodiments, the present specification discloses an illuminator window having a length that is shorter than conventional illuminator windows, enabling the window to be firmly attached to an illuminator cavity (in an endoscope tip) and thereby reducing the risk of detachment during the endoscope's operation. In embodiments, the reduction in window size enables reduction of the distance between the window and an illuminator placed in the illuminator cavity.

In an embodiment, by reducing the window size, the gap between the sides of the window and the side walls of the cavity is increased from 0.05 mm in conventional designs to approximately 0.3 mm in the present design. A larger gap enables a larger quantity of glue to be applied for adhering the sides of the window to the side walls of the cavity, causing a firmer attachment over a larger surface area.

In another embodiment, the present specification discloses an illuminator cavity which includes a support element for preventing the detachment of an illuminator window from the cavity. The reduction in window size as well as introduction of a support element enables better alignment of the window with respect to the cavity and the illuminator, and also enables placement of the window in the center of the cavity. The reduction of distance between the illuminator and the illuminator window results in an increased number of light rays emitted by the illuminator exiting the window and optimizes the illumination providing more brightness than that observed in conventional cavity/window designs.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise” “include” and “have”, and forms thereof, are not necessarily limited to members in a list with which the words may be associated.

It should be noted herein that any feature or component described in association with a specific embodiment may be used and implemented with any other embodiment unless clearly indicated otherwise.

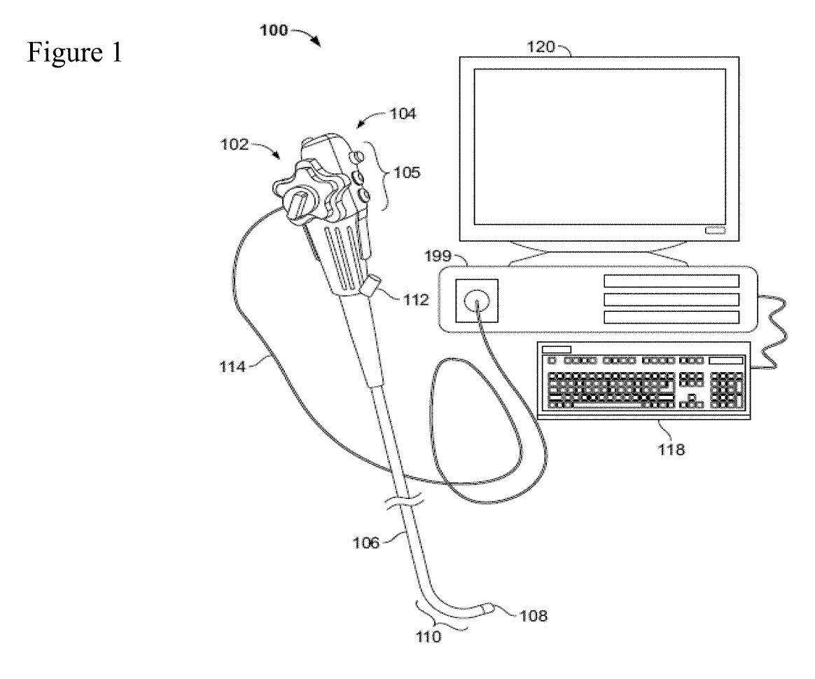

Reference is now made to FIG. 1, which shows a multiple viewing element endoscopy system 100. System 100 may include a multiple viewing element endoscope 102. Multiple viewing element endoscope 102 may include a handle 104, from which an elongated shaft 106 emerges. Elongated shaft 106 terminates with a tip section 108 which is turnable by way of a bending section 110. Handle 104 may be used for maneuvering elongated shaft 106 within a body cavity. The handle may include one or more buttons and/or knobs and/or switches 105 which control bending section 110 as well as functions such as fluid injection and suction. Handle 104 may further include at least one, and in some embodiments, one or more working channel openings 112 through which surgical tools may be inserted as well as one and more side service channel openings.

A utility cable 114, also referred to as an umbilical tube, may connect between handle 104 and a Main Control Unit 199. Utility cable 114 may include therein one or more fluid channels and one or more electrical channels. The electrical channel(s) may include at least one data cable for receiving video signals from the front and side-pointing viewing elements, as well as at least one power cable for providing electrical power to the viewing elements and to the discrete illuminators.

The main control unit 199 contains the controls required for displaying the images of internal organs captured by the endoscope 102. The main control unit 199 may govern power transmission to the endoscope's 102 tip section 108, such as for the tip section's viewing elements and illuminators. The main control unit 199 may further control one or more fluid, liquid and/or suction pump(s) which supply corresponding functionalities to the endoscope 102. One or more input devices 118, such as a keyboard, a touch screen, a voice controller and the like may be connected to the main control unit 199 for the purpose of user interaction with the main control unit 199. In the embodiment shown in FIG. 1, the main control unit 199 comprises a screen/display 120 for displaying operation information concerning an endoscopy procedure when the endoscope 102 is in use. The screen 120 may be configured to display images and/or video streams received from the viewing elements of the multi-viewing element endoscope 102. The screen 120 may further be operative to display a user interface for allowing a human operator to set various features of the endoscopy system.

Optionally, the video streams received from the different viewing elements of the multi-viewing element endoscope 102 may be displayed separately on at least one monitor (not seen) by uploading information from the main control unit 199, either side-by-side or interchangeably (namely, the operator may switch between views from the different viewing elements manually). Alternatively, these video streams may be processed by the main control unit 199 to combine them into a single, panoramic video frame, based on an overlap between fields of view of the viewing elements. In an embodiment, two or more displays may be connected to the main control unit 199, each for displaying a video stream from a different viewing element of the multi-viewing element endoscope 102. The main control unit 199 is described in U.S. patent application Ser. No. 14/263,896, entitled “Method and System for Video Processing in a Multi-Viewing Element Endoscope” and filed on Apr. 28, 2014, which is herein incorporated by reference in its entirety.

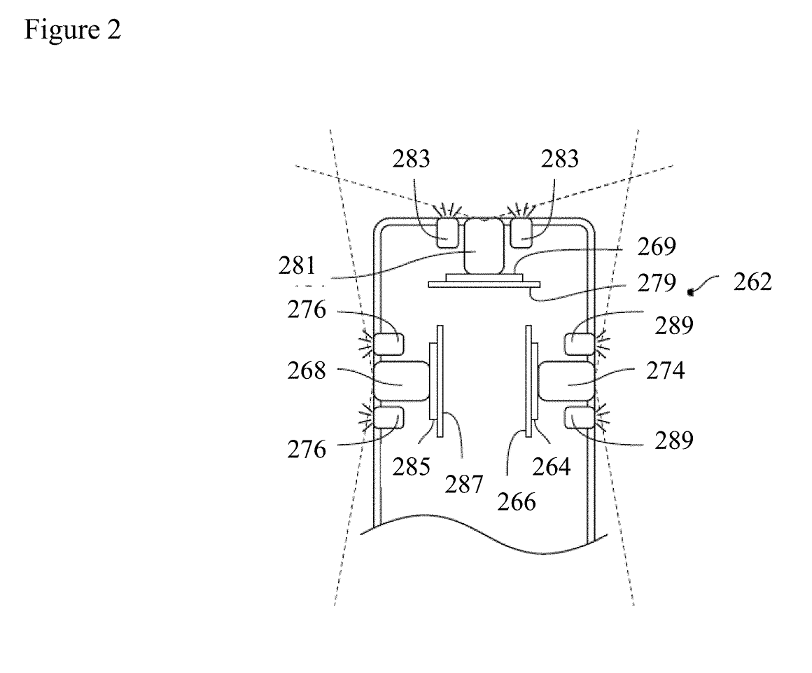

Reference is now made to FIG. 2, which shows a cross-sectional view of a tip section 262 of a multi-viewing element endoscope, according to another embodiment of the specification. Tip section 262 may include a front-pointing image sensor 269, such as a Complementary Metal Oxide Semiconductor (CMOS) image sensor. Front-pointing image sensor 269 may be mounted on an circuit board 279, which may be rigid or flexible. Circuit board 279 may include a printed circuit board (PCB), one or more integrated circuits and/or microchips on the PCB, and may be integrated into tip section 262 of the multi-viewing element endoscope. Circuit board 279 may be employed to supply front-pointing image sensor 269 with the necessary electrical power and may derive still images and/or video feeds captured by the image sensor. Circuit board 279 may be connected to a set of electrical cables which may be threaded through an electrical channel running through the elongated shaft of the endoscope. Front-pointing image sensor 269 may have a lens assembly 281 mounted on top of it for providing the necessary optics for receiving images.

Lens assembly 281 may include a plurality of lenses, static or movable, which may provide a field of view of at least 90 degrees and up to essentially 180 degrees. In one embodiment, lens assembly 281 may provide a focal length of about 3 to 100 millimeters. The term focal length may be used to refer to the distance from a lens to a sensor or may be used to refer to the distance, from the lens, over which an object remains in focus. One of ordinary skill in the art would understand what definition for focal length is being used based on the context and distances discussed.

Front-pointing image sensor 269 and lens assembly 281, with or without circuit board 279, may be jointly referred to as a “front-pointing camera”. One or more discrete front illuminators 283 may be placed next to lens assembly 281, for illuminating its field of view. In an alternate embodiment, discrete front illuminators 283 may also be attached to the same circuit board 279 upon which front-pointing image sensor 269 is mounted.

Tip section 262 may optionally include, in addition to a first side-pointing image sensor 285, a second side-pointing image sensor 264. While FIG. 2 is discussed herein with respect to an embodiment with two side-pointing image sensors, it should be understood to those of ordinary skill in the art, that, in some embodiments, only one side pointing image sensor may be used.

Referring back to FIG. 2, side-pointing image sensors 285 and 264 may include a Complementary Metal Oxide Semiconductor (CMOS) image sensor. Side-pointing image sensors 285 and 264 may be mounted on circuit boards 287 and 266, respectively, which may be rigid or flexible. Circuit boards 287 and 266 may supply side-pointing image sensors 285 and 264 with the necessary electrical power and may derive still images and/or video feeds captured by the image sensor. Circuit boards 287 and 266 may be connected to a set of electrical cables (not shown) which may be threaded through an electrical channel running through the elongated shaft of the endoscope.

In another embodiment, side-pointing image sensors 285 and 264 receive the necessary electrical power from one circuit board adapted to supply the necessary electrical power to both the sensors.

Side-pointing image sensors 285 and 264 may have lens assemblies 268 and 274, respectively, mounted on top of them for providing the necessary optics for receiving images. Lens assemblies 268 and 274 may include a plurality of lenses, static or movable, which may provide a field of view of at least 90 degrees and up to essentially 180 degrees. Side-pointing image sensors 285 and 264 and lens assemblies 268 and 274, with or without circuit boards 287 and 266, respectively, may be jointly referred to as a “side-pointing cameras”.

Discrete side illuminators 276 and 289 may be placed next to lens assemblies 268 and 274, respectively, for illuminating its field of view. Optionally, in an alternate embodiment, discrete side illuminators 276 and 289 may be attached to the same circuit boards 287 and 266 on which side-pointing image sensors 285 and 264 are mounted.

In another configuration, circuit boards 279, 287, and 266 may be a single circuit board on which front and side-pointing image sensors 269, 285, and 264, respectively, are mounted.

Front and side-pointing image sensors 269, 285, and 264 may be similar, identical or distinct in terms of, for example, field of view, resolution, light sensitivity, pixel size, focal length, focal distance and/or the like.

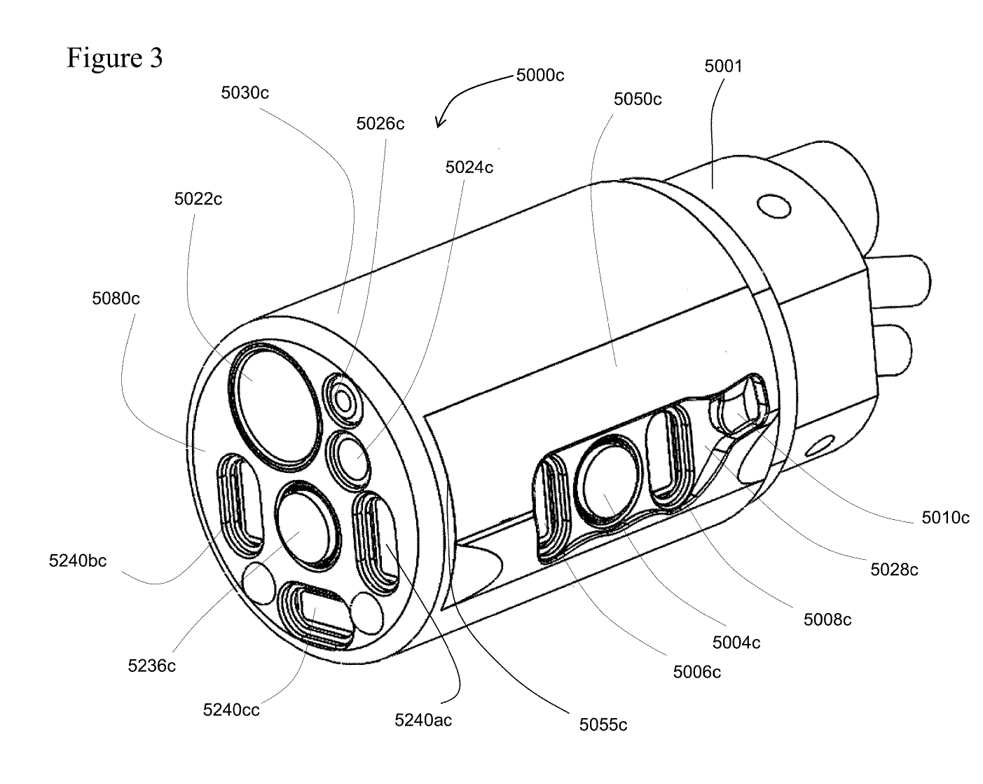

FIG. 3 shows an isometric view of a conventional multiple viewing element tip cover 5000c. Referring to FIG. 3, the multiple viewing element tip cover 5000c has a main component portion 5030c and a side removable window component 5050c located in a side panel recess or opening 5055c of the main component portion 5030c. The side removable window component 5050c is sized and configured to fit within side panel opening or recess 5055c. Main component portion 5030c is configured to cover a major portion of a tip section 5001 of an endoscope. The side removable window component 5050c, in an embodiment, is in the form of a removable panel. In an embodiment, the side removable window component 5050c comprises a flat depression 5028c which comprises a window or opening 5004c for a side optical lens assembly, optical windows 5006c, 5008c for illuminators, and a side nozzle opening 5010c for a side nozzle.

A front panel 5080c is positioned on a front end of the tip cover 5000c. The front panel 5080c comprises a window or opening 5236c for a front optical lens assembly, optical windows 5250a, 5250b and 5250c for front illuminators, a working/service channel opening 5022c, a nozzle opening 5024c and a jet opening 5026c.

It may be noted that the overall shape of the distal tip is substantially round, yet the side components including the side camera, LEDs, and jet are placed in flat depression 5028c. This flat depression 5028c is located on a surface of the side removable window component 5050c.

In an embodiment, multiple viewing element tip cover 5000c has one side removable window component 5050c on one side. In another embodiment, multi-element tip cover 5000c has more than one side removable window component on its tip side surfaces to enable easy access to the internal components in the tip section, such that a component may be removed or replaced if required without having to remove the entire tip cover 5000c.

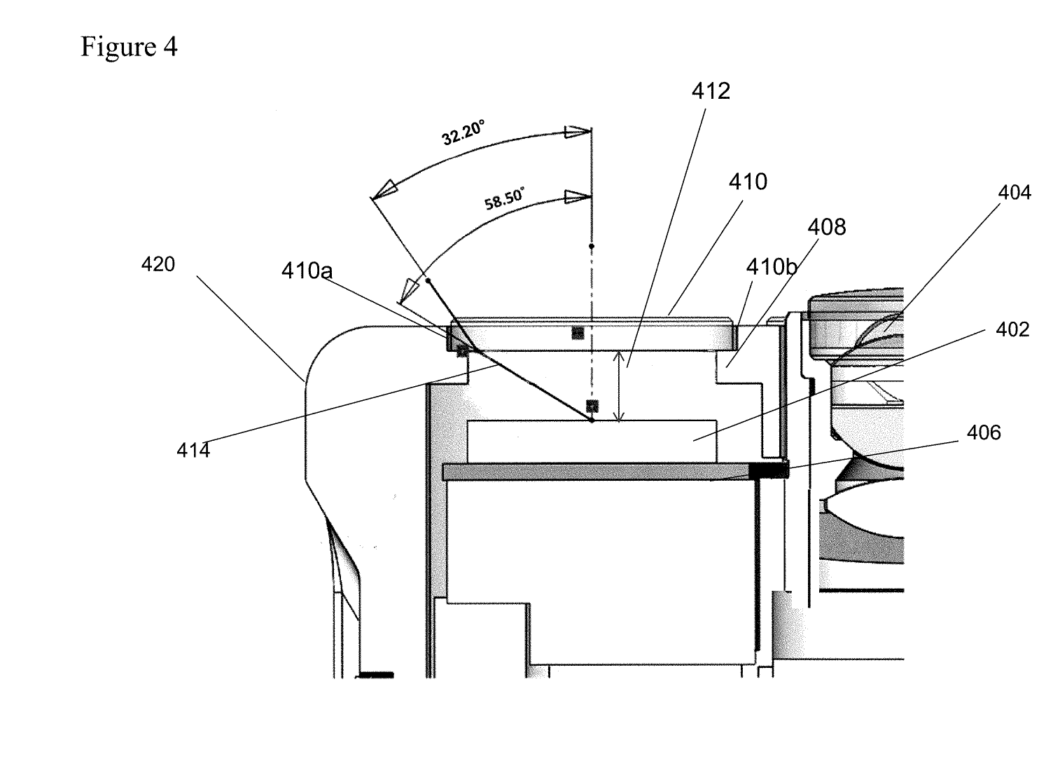

FIG. 4 illustrates a conventional illuminator coupled with an optical assembly in a tip section of an endoscope. As shown in the figure, an illuminator 402, which in an embodiment is an LED is placed adjacent to an optical assembly 404 for illuminating its field of view. The illuminator 402 is coupled with and controlled by an electronic circuit board 406; and is placed within an illuminator cavity 408. A glass illuminator window 410 seals off the cavity 408 from the surroundings. Conventionally, a gap 412 between the illuminator window 410 and the illuminator 402 ranges between 0.05 mm to 0.8 mm. Due to this gap 412 between the window 410 and the illuminator 402, there is a reduction in the intensity of light transmitted by the illuminator 402 as some of the emitted rays fall on the sides of the cavity 408 and not on the window 410, and are thereby not transmitted out of the endoscope tip. Further as shown a ray 414 emitted by the illuminator 402 bends upon transmission through the window 410 such that its wavelength reduces from 58.50 degrees to 32.20 degrees upon exiting the window 410.

The illuminator window 410 is manually placed in the cavity 408 after application of glue on its side surfaces 410a and 410b, such that the window 410 adheres to the side walls of the cavity as shown, and the corners of the window 410 rest on a surface of the cavity 408. In this conventional design the gap between the window side surfaces 410a, 410b and the side walls of the cavity 408 is ranges from approximately 0.05 mm to 0.8 mm which is extremely small for application of glue. Since the quantity of glue that can be applied in the gap is very small, the window 410 is susceptible to being detached from the tip cover 420 of the endoscope during operation or cleaning.

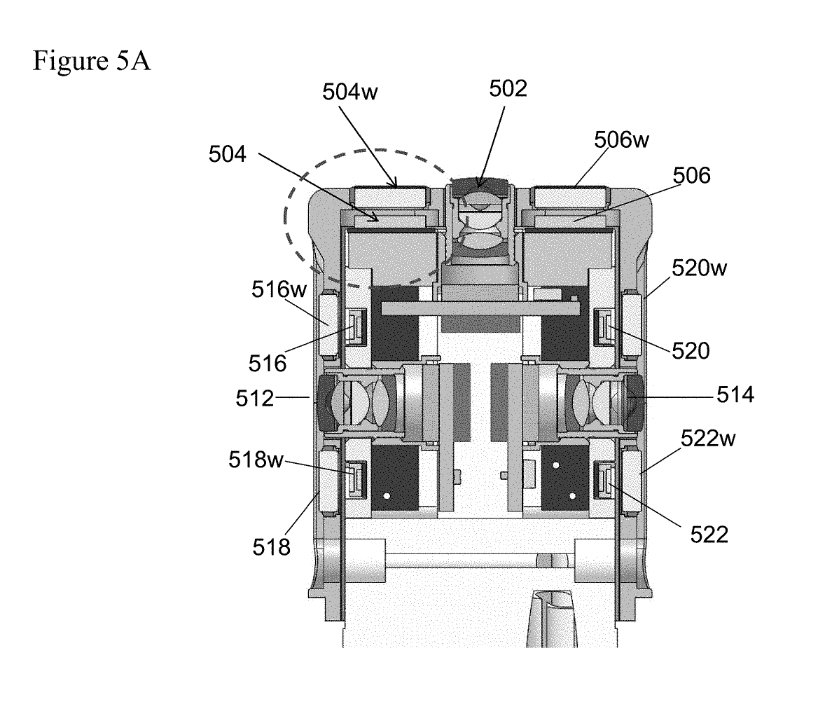

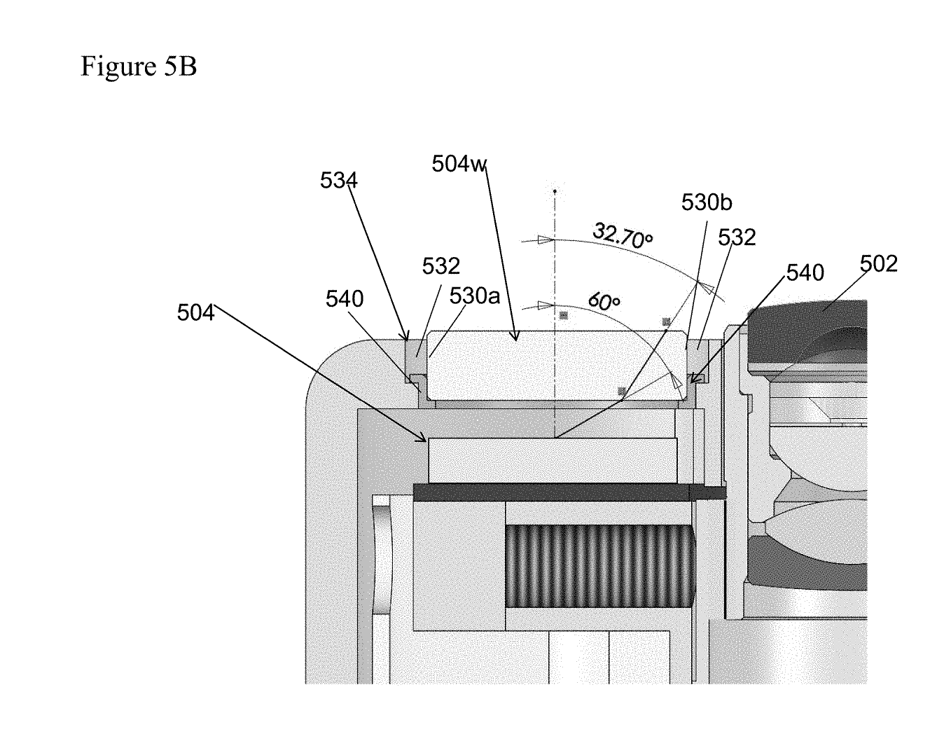

In an embodiment, the length of window 410 is reduced to increase the gap and hence the quantity of glue between the window sides and the cavity walls, thereby adhering the window more strongly within the tip cover 420. FIG. 5A illustrates illuminator windows affixed to illuminator cavities in an endoscope tip, in accordance with an embodiment of the present specification. As shown a front optical assembly 502 is flanked on both sides by illuminators 504 and 506, which are covered by illuminator windows 504w and 506w respectively. Similarly, side pointing optical assemblies 512 and 514 are flanked on both sides by illuminators 516, 518 and 520, 522 respectively. Illuminator windows 516, 518, 520 and 522 are covered with glass windows 516w, 518w, 520w and 522w respectively. Each illuminator is placed in an illuminator cavity which is sealed off by affixing an illuminator window to it, as shown in FIG. 4 above.

FIG. 5B illustrates a close up cross sectional view of the illuminator window 504w affixed to an illuminator cavity shown in FIG. 5A. Referring simultaneously to FIGS. 5A and 5B, gap 532 between side walls 530a, 530b of illuminator window 504w and illuminator cavity 534 is increased to approximately 0.3 mm by reducing the length of the illuminator window 504w. In embodiments, the illuminator cavity may be of any shape, such as a square, a rectangle, an oval, or oblong, but not limited to such shapes. In an embodiment, the illuminator cavity is appropriately shaped to house the illuminator and/or corresponding circuit board. Further, a window support element 540 is attached to the sidewalls of the cavity 534 for providing extra support to the window sidewalls 530a, 530b respectively. In an embodiment the window support element 540 is a metal ring that allows the placement of the window 504w in a desired location within the cavity 534. In various embodiments, the window support element 504w fits in the cavity 534 due to a reduction in the size of window 504w. Hence, the distance between an illuminator and the corresponding illuminator window may be controlled by using the window support element. The reduction in window size enables a reduction of the distance between the window 504w and the illuminator 504. By reducing the distance between the illuminator, reduction in the intensity of light transmitted by the illuminator 504 does not occur. Even though the new design of the illuminator window has been described with respect to front window 504w, it is apparent that the same design may be applied to the side illuminator windows as well.

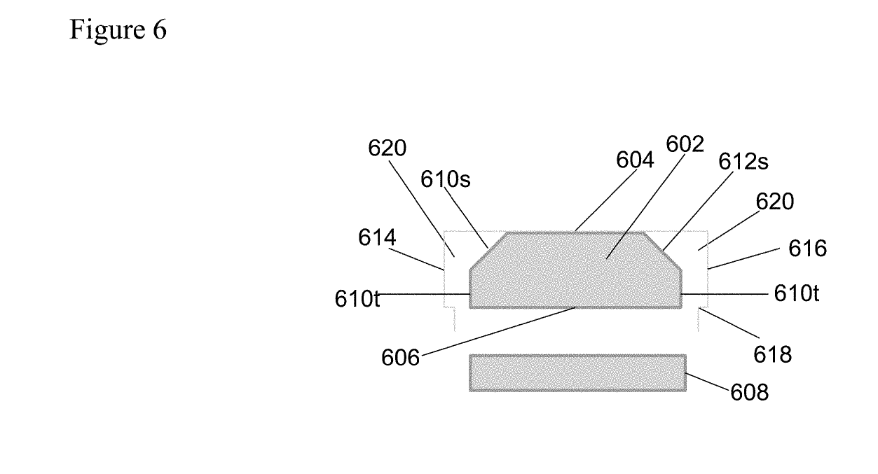

In an embodiment, an illuminator window is truncated at a distal end that faces away from the illuminator such that length of the side facing outside of the endoscope's tip is less than the length of the side facing the illuminator. This shape results in a larger gap between the side walls of the illuminator cavity and the sides of the window for application of a larger quantity of glue, thereby bonding the window tightly with the cavity walls, and preventing it from falling off the cavity. FIG. 6 illustrates a block diagram of an illuminator window affixed to an illuminator cavity in an endoscope tip, in accordance with an embodiment of the present specification. Illuminator window 602 comprises an outer surface 604 facing outside the endoscope tip (not shown), an inner surface 606 facing an illuminator 608, a left slanted side portion 610s extending into a left straight side portion 610t and a right slanted side portion 612s extending into a right straight side portion 612t. Window 602 is affixed to side walls 614 and 616 of illuminator cavity 618 by means of glue filled in the gap 620 between the sides of the window 602 and the cavity walls 614, 616. The slanted shape of the sides 610s and 612s provides a larger volume between the sidewalls 614, 616 and the window 602 as compared to prior art illuminator window designs.

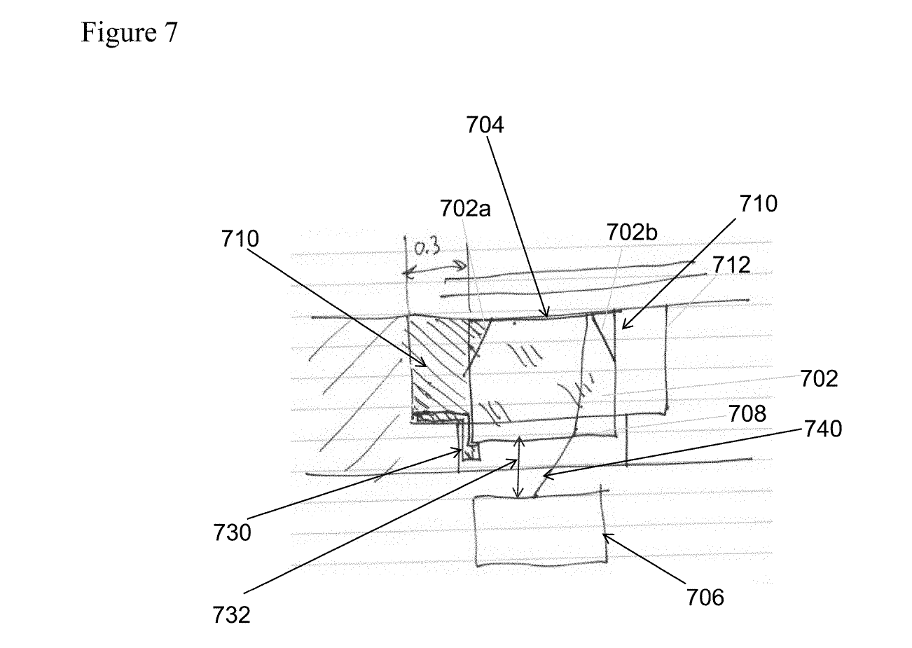

FIG. 7 illustrates a cross sectional view of an illuminator window affixed to an illuminator cavity, in accordance with an embodiment of the present specification. As shown in the figure, the sides of illuminator window 702 are truncated at a distal end creating two slanting sides 702a and 702b, and making surface 704 facing away from the illuminator 706 shorter in length than surface 708 facing the illuminator 706. This truncation of the window 702 increases the gap 710 between the sides of the window 702 and sidewalls 712a, 712b of the illuminator cavity 712, so that a larger quantity of glue may be applied in the gap resulting in a firmer attachment of the window 702 with the cavity 712. In an embodiment, a window support element 730 is fitted into the cavity 712 for providing extra support to the window 702. In an embodiment the window support element 730 is a metal ring that allows the placement of the window 702 in a desired location within the cavity 712. Hence, the distance 732 between an illuminator and the corresponding illuminator window may be reduced by reducing the window size and by using the window support element. By reducing the distance 732 between the illuminator to approximately 0.4 mm, reduction in the intensity of light transmitted by the illuminator 706 does not occur. A ray of light 740 emitted by the illuminator 706 bends and changes its speed and wavelength upon entering the window 702, as shown in the figure.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. An endoscope tip comprising an illuminator window for covering an illuminator cavity comprising an illuminator coupled with a circuit board installed in the endoscope tip, the illuminator window comprising a distal long surface facing away from the illuminator, a proximal long surface facing towards the illuminator, and a first and a second side surface, the cavity comprising a first side wall positioned at a predefined distance from the first side surface of the window creating a first gap and a second side wall positioned at a predefined distance from the second side surface of the window creating a second gap, the first side surface of the window being attached to the first sidewall by application of glue in the first gap and the second side surface of the window being attached to the second sidewall by application of glue in the second gap, wherein the width of the first and the second gaps is greater than 0.8 mm and the distance between the illuminator and the window is less than or equal to 0.4 mm.

2. The endoscope tip of claim 1 wherein the illuminator window is rectangular in shape, wherein the length of the distal surface is equal to the length of the proximal surface, and the length of the first side surface is equal to the length of the second side surface.

3. The endoscope tip of claim 1 wherein the length of the distal and proximal surfaces is within a predetermined range.

4. The endoscope tip of claim 1 wherein the width of the first and the second gap is 0.3 mm.

5. The endoscope tip of claim 1 wherein the width of the first and the second gap is within a predetermined range.

6. The endoscope tip of claim 1 wherein the cavity comprises a window support element for enabling the window to be placed in the center of the cavity.

7. The endoscope tip of claim 6 wherein the window support element is a metal ring having a predefined diameter.

8. The endoscope tip of claim 6 wherein the window support element enables placement of the illuminator window at a predefined distance from the illuminator.

9. The endoscope tip of claim 1 wherein the distal long surface of the window is truncated to create a first slanting side and a second slanting sides, thereby increasing the width of the first and the second gap respectively, the length of the truncated distal surface being shorter than the length of the proximal long surface.

10. The endoscope tip of claim 9 wherein the increased width of the first and the second gap is within a predetermined range.

11. An illuminator window for covering an illuminator cavity comprising an illuminator coupled with a circuit board installed in an endoscope tip, the illuminator window comprising a distal surface facing away from the illuminator, a proximal surface facing towards the illuminator, length of the proximal surface being greater than length of the distal surface, and a first and a second slanting side surface, the cavity comprising a first side wall positioned at a predefined distance from the first side surface of the window creating a first gap and a second side wall positioned at a predefined distance from the second side surface of the window creating a second gap, the first side surface of the window being attached to the first sidewall by application of glue in the first gap and the second side surface of the window being attached to the second sidewall by application of glue in the second gap, wherein the width of the first and the second gaps is greater than 0.8 mm and the distance between the illuminator and the window is less than or equal to 0.4 mm.

12. The illuminator window of claim 11 wherein the distal surface is parallel to the proximal surface.

13. The illuminator window of claim 11 wherein the window is shaped as a parallelogram.

14. The illuminator window of claim 11 wherein the length of the first slanting side surface is equal to the length of the second slanting side surface, said length being smaller than lengths of the distal and proximal surfaces.

15. The illuminator window of claim 11 wherein the length of the distal surface and proximal surface are within predetermined ranges.

16. The illuminator window of claim 11 wherein the width of the first and the second gap is 0.3 mm.

17. The illuminator window of claim 11 wherein the width of the first and the second gap are within predetermine ranges.

18. The illuminator window of claim 11 wherein the cavity comprises a window support element for enabling the window to be placed in the center of the cavity.

19. The illuminator window of claim 18 wherein the window support element is a metal ring having a diameter within a predetermined range.

20. The illuminator window of claim 18 wherein the window support element enables placement of the illuminator window at a predefined distance from the illuminator.
